(19) United States
(12) Patent Application Publication
(10) Pub. No.: US 2006/0147526 A1
(43) Pub. Date: Jul. 6, 2006

(54) Pharmaceutical formulations comprising ß-2 adrenoreceptor agonists and xanthines

(75) Inventors: Amar Lulla, Maharashtra (IN); Geen Malhotra, Maharashtra (IN)

Correspondence Address: STEVENS DAVIS MILLER & MOSHER, LLP, 1615 L STREET, NW, SUITE 850, WASHINGTON, DC, 20036, US

(73) Assignee: CIPLA LIMTED, Mumbai Central, Mumbai (IN)

(21) Appl. No.: 10/536,353

(22) PCT Filed: Dec. 1, 2003
(86) PCT No.: PCT/GB03/05232

§ 371 Date: Oct. 31, 2005

(30) Foreign Application Priority Data
Nov. 29, 2002 (GB) 02279487

(51) Int. Cl. A61K 31/522; A61K 9/22

(52) U.S. Cl. 424/468; 514/263.31

(57) ABSTRACT

A pharmaceutical formulation comprising (i) a sustained release layer comprising at least one xanthine, and (ii) an immediate release layer comprising at least one β-2 adrenoreceptor agonist selected from the group consisting of long acting and short acting β-2 adrenoreceptor agonists.

The present invention is concerned with pharmaceutical formulations comprising anti-asthmatics, such as β-2 adrenoreceptor agonists and xanthines, useful in the prophylaxis and treatment of respiratory diseases.

The path physiology of asthma or related disorders involves bronchoconstriction resulting from bronchial smooth muscle spasm and airway inflammation with mucosal edema. Treatment of asthma and other related disorders have been known to employ β-2 agonists, also known as β-2 adrenoreceptor agonists. Such β-2 adrenoreceptor agonists are known to provide a bronchodilator effect to patients, resulting in relief from the symptoms of breathlessness. More particularly, β-2 adrenoreceptor agonists have been shown to increase the conductance of potassium channels in airway muscle cells, leading to membrane hyperpolarization and relaxation.

It has also been seen that a subgroup of β-2 adrenoreceptor agonists can be described as short acting, whilst a further subgroup of β-2 adrenoreceptor agonists can be described as long acting. The mechanism of anti-asthmatic action of the short acting subgroup is linked to the direct relaxation of airway smooth muscles and consequent bronchodilation. Drugs belonging to this short acting subgroup have quicker onset of action (within 1-15 minutes) and produce a bronchodilation that lasts for about 2-6 hours. Such drugs act as “rescue” medication i.e. where immediate relief is required. The long acting subgroup of drugs act via a similar mechanism i.e. relaxation of airway smooth muscles and consequent bronchodilation. Drugs of this long acting subgroup may have delayed onset of action, but have a longer duration of action, so are used for long-term regular treatment of reversible airways obstruction in asthma.

Short acting β-2 adrenoreceptor agonists include lev-albuterol, R,R-formoterol, metaproternol sulfate, pirbuterol acetate, bitolterol mesylate, procaterol and the like. Long acting β-2 adrenoreceptor agonists include salmeterol, bambuterol and formoterol. For example, it has been reported that inhalation of salmeterol provides persistent bronchodilation lasting over 12 hours. The extended side chain on salmeterol renders it greatly lipophilic. This lipophilicity regulates the diffusion rate away from the receptor by determining the degree of partitioning in the lipid bilayer of the membrane. Subsequent to binding the receptor, the less lipophilic, short acting agonists are rapidly removed from the receptor environment by diffusion in aqueous phase. Unbound salmeterol, by contrast, persists in the membrane and only slowly dissociates from the receptor environment.

There is additionally a class of β-2 adrenoreceptor agonists recognized to be intermediate acting β-2 adrenoreceptor agonists, and these can include drugs such as terbutaline, salbutamol and fenoterol.

Xanthines have two distinct actions in the airways of patients with reversible obstruction, namely smooth muscle relaxation (i.e., bronchodilation) and suppression of the response of the airways to stimuli (i.e., non-bronchodilator prophylactic effects). Examples of xanthines include theophylline, theobromine, aminophylline, doxophylline, enprofylline and the like. In particular, theophylline (a methylxanthine) has proven efficacy as a bronchodilator in asthma and has been considered first-line therapy.

Bronchodilation by theophylline is mediated by the inhibition of two isozymes of phosphodiesterase, that is PDE III, and to a lesser extent, PDE IV. Non-bronchodilator prophylactic actions of theophylline are probably mediated through one or more different molecular mechanisms, that do not involve inhibition of PDE III or antagonism of adenosine receptors. For example, theophylline increases the force of contraction of diaphragmatic muscles and this action appears to be due to enhancement of calcium uptake through an adenosine-mediated channel.

It has also been reported that slow release theophylline improves nocturnal asthma. Typically, such extended release formulations of theophylline allow twice-daily dosing.

Examples of prior art slow release or sustained release matrices include those in U.S. Pat. Nos. 2,809,916, 3,062,720, 3,577,514 and 3,909,444.

U.S. Pat. No. 2,809,916 describes the manufacture of sustained release tablets formed from granules of medicament and enteric, water-insoluble excipients (including cellulose acetate phthalate) by repeatedly mixing, drying and crushing the medicament and excipients.

U.S. Pat. No. 3,062,720 describes the formation of sustained release tablets from medicament, insoluble solid fatty materials and fillers. Sustained release is accomplished by maintaining a “solubility factor” within a specified range and the solubility factor in turn is dependent on the solubility and weight percent of each of the ingredients.

U.S. Pat. No. 3,577,514 describes a sustained release tablet comprising up to 70% active ingredient, 15 to 50% hydrophobic dissolution retardant, such as natural and synthetic waxes, resins and plastics, 0.1 to 5% acid-insoluble release agent, such as cellulose acetate phthalate, 5 to 15% water soluble binder, such as polyvinyl pyrrolidone and, optionally, lubricants such as talc and magnesium stearate. Theophylline is among the active materials used in the tablet.

U.S. Pat. No. 3,909,444 describes microcapsules, which contain a continuous matrix of water soluble polymeric material (such as polyvinyl pyrrolidone) in which finely divided particles of active are dispersed. Some of the active particles are enteric coated (including esters and half esters of cellulose acetate phthalate) and the microcapsules are coated with a water-soluble polymer (such as polyvinyl acetate).

Further examples of theophylline release systems are set forth in U.S. Pat. Nos. 3,109,775, 4,261,970, 4,415,547 and 4,465,660.

U.S. Pat. No. 3,109,775 describes a tablet for release of theophylline. The tablet may be composed of the theophylline medicament having a retardant coating. The theophylline is coated onto a sugar/starch pellet by means of an adhesive such as cellulose acetate phthalate or polyvinyl pyrrolidone. The pellet may then be coated, for example with cellulose acetate phthalate, which will not dissolve in stomach juices.

U.S. Pat. No. 4,261,970 describes a sustained release theophylline granule containing a metal salt of a fatty acid and ethyl cellulose.

U.S. Pat. No. 4,415,547 describes a sustained release tablet composed of encapsulated pellets and a tableting mixture. The pellets are a sugar-starch bead coated with a first coat of 75 to 90% theophylline and 1 to 35% polyvinyl pyrrolidone and a second coat of from 1 to 10% polyvinyl pyrrolidone, 1 to 60% ethyl cellulose and 30 to 98% dusting powder.

U.S. Pat. No. 4,465,660 describes nondisintegrating theophylline tablets, which remain intact during dissolution over an extended period. The tablets are formed of crystalline pulverulent theophylline without tableting aid or other carriers.

Combination of xanthines with intermediate acting β-2 adrenoreceptor agonists is also known from the prior art as follows.

FR 2390164 is concerned with the combination of a methylxanthine, such as theophylline or aminophylline, with a β-2 agonist, such as salbutamol, terbutaline, soterenol or fenoterol.

DE 3511236 relates to the combination of theophylline and terbutaline, where pellets of the active ingredients are respectively formed and filled into capsules.

International Journal of Clinical Pharmacology, Therapy and Toxicology (vol. 25, no. 10, 1987, pages 558-564) investigates combination therapy with theophylline and terbutaline, with theophylline and terbutaline respectively administered as sustained release tablets.

EP 0173928A is concerned with a controlled release coated pharmaceutical preparation comprising a drug tablet and a coating applied thereon, where the coating essentially consists of a film-forming polymer which is insoluble in water and gastrointestinal fluids and a water-soluble pore creating material being randomly distributed in the polymer. The preparation is characterised in that the pore creating material partially or totally consists of a drug active substance in sufficient amounts to produce a pharmacological or therapeutic effect. In one embodiment, the core can comprise theophylline or a theophylline salt, and the pore creating material is a beta-2-stimulant, such as salbutamol or terbutaline.

Despite the large numbers of known treatments of respiratory diseases as discussed above, there continues to exist a clinical need for therapies of respiratory diseases which exhibit advantageous profiles of action. To this end, it has surprisingly been found that a combination of a β-2 adrenoreceptor agonist selected from long acting and short acting β-2 adrenoreceptor agonists, with xanthines, provides an enhanced, synergistic therapeutic effect in terms of treatment of respiratory diseases, such as bronchoconstriction, mucous secretions and related disorders. Also, such combination therapy is an extremely patient-friendly combination, which results in enhanced patient compliance and better control of respiratory diseases, such as asthma, compared to known combinations.

In particular, it has surprisingly been found that a combination of a β-2 adrenoreceptor agonist selected from long acting and short acting β-2 adrenoreceptor agonists, with xanthines, provides an enhanced, synergistic therapeutic effect in terms of treatment of respiratory diseases, such as nocturnal asthma and other related disorders.

Accordingly, it is an object of the present invention to provide methods of treating nocturnal asthma. It is a further object of the present invention to provide a pharmaceutical formulation for the time-specific delivery of pharmaceutically active agents for the treatment of nocturnal asthma.

According to the present invention, therefore, there is provided a pharmaceutical tablet formulation comprising (i) a sustained release layer comprising at least one xanthine, and (ii) an immediate release layer comprising at least one β-2 adrenoreceptor agonist selected from the group consisting of long acting and short acting β-2 adrenoreceptor agonists.

Suitably in pharmaceutical products or formulations according to the present invention, a long acting β-2 adrenoreceptor agonist is selected from the group consisting of, salmeterol, bambuterol and formoterol, or a pharmaceutically acceptable salt, solvate or physiologically functional derivative thereof. Preferably a long acting β-2 adrenoreceptor agonist employed according to the present invention is bambuterol hydrochloride.

Bambuterol, the bis-dimethyl carbamate pro drug of terbutaline, namely 5-[2-(tert-butylamino)-1-hydroxyethyl]-m-phenylene-bis(dimethylcarbamate), or a pharmaceutically acceptable salt thereof, is described in the European patent EP 43807. Bambuterol has been used in the treatment of asthmatic patients, with no observable effect on the lipoprotein metabolism having been reported. Substantially as hereinbefore described, bambuterol is a prodrug of the β-2 adrenoreceptor agonist terbutaline, and as such gives a significantly prolonged duration of action, for example 24 hours versus 8 hours for conventional terbutaline tablets, or 12 hours for terbutaline slow release tablets. It is especially preferred to use the hydrochloride salt of bambuterol.

Suitably in pharmaceutical formulations according to the present invention, a short acting β-2 adrenoreceptor agonist is selected from the group consisting of levalbuterol, R,R-formoterol, metaprolol sulfate, pirbuterol acetate, bitolterol mesylate, procaterol, and the like.

Suitably in pharmaceutical formulations according to the present invention, a xanthine is selected from the group consisting of, theophylline, theobromine, aminophylline, doxophylline and enprofylline, or a pharmaceutically acceptable salt, solvate or physiologically functional derivative thereof. Preferably a methylxanthine employed according to the present invention is theophylline.

In a preferred embodiment according to the present invention the formulation preferably further comprises a water soluble or water swellable polymeric release material, which preferably delays the release of the one or more xanthines from the sustained release layer for a predetermined period of time, with immediate release of the β-2 adrenoreceptor agonist. Typically, the sustained release layer is prepared by admixing the xanthine with suitable excipients required for the sustained release layer, the resulting mixture is then blended and wet granulated with a portion of the water soluble or water swellable polymeric release material, the resulting granulate is sized and typically mixed with further water soluble or water swellable polymeric release material and a suitable lubricant. The resulting mixture can then suitably be compressed using standard techniques.

Suitably a xanthine containing layer of a pharmaceutical formulation according to the present invention may be formulated so as to allow the release of the one or more xanthines therefrom in a still further controlled or sustained release manner, subsequent to the desired lag-time provided by the inclusion of a water soluble or water swellable polymeric material which is preferably employed according to the present invention. It will be appreciated, therefore, that conventional excipients may be employed in a xanthine containing layer of a formulation according to the present invention; alternatively, excipients which are capable of forming a sustained release matrix system may be used in the xanthine containing layer.

Preferably in a formulation according to the present invention, theophylline, after oral administration, can be released in a sustained manner independent of pH and bambuterol can be released immediately. It has been seen that tablets according to the present invention produce relatively uniform blood levels of theophylline over extended periods of therapy, suitably with oral administration at intervals of about twelve hours. A sustained release can thus be achieved by means of a polymeric matrix employed in the tablet formulation substantially as hereinbefore described and a tablet according to the present invention tends to swell and slowly erode, rather than disintegrate. Such erosion proceeds for an extended or sustained period of time, with release of a xanthine, such as theophylline, by a diffusion process substantially as hereinbefore described. A tablet as provided by the present invention disintegrates into particles only after several hours.

A tablet according to the present invention comprises a combination of materials, including for example one or more water soluble or swellable hydrophilic or lipophillic gel forming polymers, diluents and optionally a hydrophobic lubricant. The above materials are combined with a xanthine, such as theophylline in the following proportions, to achieve the beneficial steady or sustained release characteristics of the present invention:
(a) 40 to 55 weight % one or more xanthines (in particular theophylline);
(b) 10 to 25 weight % water soluble or water swellable hydrophilic or lipophillic gel forming polymers;
(c) 10 to 30 weight % diluents; and
(d) 1 to 5 weight % binders.

A water soluble or water swellable polymer employed in a pharmaceutical formulation according to the present invention swells and dissolves thereby permitting controlled drug dissolution as the gastro-intestinal fluids penetrate and erode a tablet according to the present invention. In effect, a tablet of the invention swells and slowly erodes releasing the drug by a diffusion process. Only after several hours does the tablet disintegrate into particles. In in-vitro tests using gastro-intestinal fluids, a xanthine such as theophylline, is characteristically released at a predetermined rate, regardless of pH.

A water soluble or water swellable polymer or gel forming polymer employed in a pharmaceutical formulation according to the present invention, may be polyvinylpyrrolidone, or a cellulose derivative, such as hydroxypropyl methyl cellulose, methyl cellulose, sodium carboxy methyl cellulose, cross linked carboxy methyl cellulose, hydroxy ethyl cellulose, hydroxy propyl cellulose and the like. A preferred polymeric material employed in a pharmaceutical formulation according to the present invention comprises hydroxypropyl methyl cellulose and polyvinylpyrrolidone.

Suitably, a diluent or bulking agent should be selected to provide an increase in tablet size. The artisan can utilize known methods to select a bulking agent, which provides hardness, friability and disintegration time required for pharmaceutical usage. A preferred diluent is lactose. Various forms of lactose are appropriate for such formulations, including anhydrous, hydrous and spray dried forms. The most desired form of lactose for use according to the present invention can be selected based on desired dissolution, content uniformity, hardness, friability and disintegration time. The artisan can use known techniques to achieve the desired physical properties.

The artisan may further select appropriate dry binders using known methods. Most preferably, dry binders are starch and microcrystalline cellulose; however, other appropriate dry binders may be selected. Microcrystalline cellulose may be in a granular form.

A tablet formulation according to the present invention may also include a hydrophobic lubricant. The artisan can select an appropriate lubricant to prevent sticking and picking of the tablets to the compression tooling. Suitable lubricants include talc, fatty acids, salts of fatty acids, mineral oil, and hydrogenated vegetable oils. An example of a suitable fatty acid material is stearic acid or its magnesium salt. The most preferred lubricant is magnesium stearate.

A sustained release source of one or more xanthines employed in a formulation according to the present invention can comprise about 50% theophylline, about 15% hydroxypropyl methyl cellulose, about 1-5% polyvinylpyrrolidone and about 0-2% of a lubricant.

The present invention further comprises a process of preparing a pharmaceutical formulation substantially as hereinbefore described, which comprises providing at least one xanthine and at least one β-2 adrenoreceptor agonist selected from the group consisting of long acting and short acting β-2 adrenoreceptor agonists, formulating the xanthine so as to provide the sustained release layer of the formulation and formulating the β-2 adrenoreceptor agonist so as to provide the immediate release layer of the formulation.

Suitably the sustained release layer comprising a xanthine, such as theophylline, may be prepared by mixing the theophylline with polymer and diluents. The resulting mixture may then be blended and wet granulated with a portion of a film former, such as polyvinylpyrrolidone. The granulate may then be sized through a sieve of optimum mesh, mixed with the remaining polymer, and a lubricant. The resulting mixture may then be compressed using a standard rotary tablet press.

It is known from the literature that direct compression processes or dry granulated processes for preparing solid oral formulations can result in undesired poor dose uniformity. Preferably the immediate release layer comprising the β-2 adrenoreceptor agonist, such as bambuterol, is prepared by mixing bambuterol with the diluents, blending well and mixing with a lubricant followed by compressing. The immediate release layer may alternatively be prepared by using wet granulation processes or fluid bed granulation processes to achieve homogenous distribution of a drug within the formulation.

The present invention further provides use of at least one long acting β-2 adrenoreceptor agonist selected from the group consisting of long acting and short acting β-2 adrenoreceptor agonists, and at least one xanthine, in the manufacture of a medicament for the treatment of respiratory disease, wherein said medicament comprises (i) a sustained release layer comprising said xanthine, and (ii) an immediate release layer comprising said β-2 adrenoreceptor agonist.

The present invention further provides a method of administering to a subject in need of treatment a pharmaceutical formulation substantially as hereinbefore described and in particular a time-specific controlled or sustained release formulation which can typically be administered in the evening, and which permits or achieves delivery of pharmaceutically active agents effective for the treatment of a specific pathology to be treated, for example over 24 hours, and as such is particularly suited for the treatment of nocturnal asthma.

The tablets of the invention are orally administered in the amounts necessary to achieve a particular blood level. Once the blood level is achieved, it can be maintained by repeated oral administration of the tablet at a dose interval of 12 hours. An optimum dosage size may be determined by observing the therapeutic results achieved and the side effects encountered and/or by blood serum analysis.

The present invention will now be further illustrated by the following examples, which do not limit the scope of the invention in any way.

### EXAMPLE I

| Ingredients | Mg/Tablet |
|---|---|
| First Layer | |
| Theophylline | 200 |
| Lactose | 39 |
| Microcrystalline cellulose | 20 |
| Hydroxypropylmethylcellulose K4M | 20 |
| Hydroxypropylmethylcellulose K15M | 12 |
| Poly vinyl pyrollidone k 30 | 5 |
| Distilled water | Qs. |
| Magnesium stearate | 1.18 |
| Second Layer | |
| Bambuterol Hydrochloride | 10 |
| Starch | 20 |
| Lactose | 42.5 |
| Microcrystalline cellulose | 30 |
| Poly vinyl pyrollidone k 30 | 2.0 |
| Distilled water | Qs. |
| Magnesium stearate | 0.42 |

Procedure:

A first layer comprising theophylline was prepared by mixing the theophylline with polymer and diluents. The resulting mixture was then blended and wet granulated with a portion of the film former, polyvinylpyrrolidone. The granulate was then sized through a sieve of optimum mesh, mixed with the remaining polymer, and the lubricant. The resulting mixture was then compressed using a standard rotary tablet press.

A second layer comprising bambuterol was prepared by mixing bambuterol with the diluents, blended well and mixed with the lubricant and compressed. (The second layer may alternatively be prepared by using wet granulation process or fluid bed granulation process to achieve homogenous distribution of the drug within the formulation.)

### EXAMPLE II

| Ingredients | Mg/Tablet |
|---|---|
| First Layer | |
| Theophylline | 200 |
| Lactose | 30 |
| Microcrystalline cellulose | 30 |
| Hydroxypropylmethylcellulose K4M | 5 |
| Hydroxypropylmethylcellulose K100M | 10 |
| Poly vinyl pyrollidone k 30 | 5 |
| Distilled water | Qs. |
| Magnesium stearate | 1.12 |
| Second Layer | |
| Bambuterol Hydrochloride | 10 |
| Starch | 20 |
| Lactose | 45 |
| Microcrystalline cellulose | 30 |
| Poly vinyl pyrollidone k 30 | 2.0 |
| Distilled water | Qs. |
| Magnesium stearate | 0.43 |

Procedure: Same as described in Example-I

### EXAMPLE III

| Ingredients | Mg/Tablet |
|---|---|
| First Layer | |
| Theophylline | 200 |
| Lactose | 25 |
| Microcrystalline cellulose | 35 |
| Hydroxypropylmethylcellulose K4M | 25 |
| Hydroxypropylmethylcellulose K15M | 10 |
| Poly vinyl pyrollidone k 30 | 5 |
| Distilled water | Qs. |
| Magnesium stearate | 1.2 |
| Second Layer | |
| Bambuterol Hydrochloride | 10 |
| Starch | 25 |
| Lactose | 55 |
| Microcrystalline cellulose | 20 |
| Poly vinyl pyrollidone k 30 | 2.0 |
| Distilled water | Qs. |
| Magnesium stearate | 0.45 |

Procedure: Same as described in Example-I

### EXAMPLE IV

| Ingredients | Mg/Tablet |
|---|---|
| First Layer | |
| Theophylline | 200 |
| Lactose | 40 |
| Microcrystalline cellulose | 20 |
| Hydroxypropylmethylcellulose K4M | 20 |
| Hydroxypropylmethylcellulose K15M | 10 |
| Poly vinyl pyrollidone k 30 | 5 |
| Distilled water | Qs. |
| Magnesium stearate | 1.18 |
| Second Layer | |
| Bambuterol Hydrochloride | 10 |
| Starch | 25 |
| Lactose | 37 |
| Microcrystalline cellulose | 38 |
| Poly vinyl pyrollidone k 30 | 2.0 |
| Distilled water | Qs. |
| Magnesium stearate | 0.45 |

Procedure: Same as described in Example-I

### EXAMPLE V

| Ingredients | Mg/Tablet |
|---|---|
| First Layer | |
| Aminophylline | 225 |
| Lactose | 39 |
| Microcrystalline cellulose | 20 |
| Hydroxypropylmethylcellulose K4M | 20 |
| Hydroxypropylmethylcellulose K15M | 12 |
| Poly vinyl pyrollidone k 30 | 5 |
| Distilled water | Qs. |
| Magnesium stearate | 1.28 |
| Second Layer | |
| Bambuterol Hydrochloride | 10 |
| Starch | 20 |
| Lactose | 42.5 |
| Microcrystalline cellulose | 30 |
| Poly vinyl pyrollidone k 30 | 2.0 |
| Distilled water | Qs. |
| Magnesium stearate | 0.42 |

Procedure:

A first layer comprising aminophylline was prepared by mixing the theophylline with polymer and diluents. The resulting mixture was then blended and wet granulated with a portion of the film former, polyvinylpyrrolidone. The granulate was then sized through a sieve of optimum mesh, mixed with the remaining polymer, and the lubricant. The resulting mixture was then compressed using a standard rotary tablet press.

A second layer comprising bambuterol was prepared by mixing bambuterol with the diluents, blended well and mixed with the lubricant and compressed. (The second layer may alternatively be prepared by using wet granulation process or fluid bed granulation process to achieve homogenous distribution of the drug within the formulation.)

### EXAMPLE VI

| Ingredients | Mg/Tablet |
|---|---|
| First Layer | |
| Dyphylline | 300 |
| Lactose | 30 |
| Microcrystalline cellulose | 30 |
| Hydroxypropylmethylcellulose K4M | 5 |
| Hydroxypropylmethylcellulose K100M | 10 |
| Poly vinyl pyrollidone k 30 | 5 |
| Distilled water | Qs. |
| Magnesium stearate | 1.52 |
| Second Layer | |
| Bambuterol Hydrochloride | 10 |
| Starch | 20 |
| Lactose | 45 |
| Microcrystalline cellulose | 30 |
| Poly vinyl pyrollidone k 30 | 2.0 |
| Distilled water | Qs. |
| Magnesium stearate | 0.43 |

Procedure: Same as described in Example-I

### EXAMPLE VII

| Ingredients | Mg/Tablet |
|---|---|
| First Layer | |
| Dyphylline | 300 |
| Lactose | 25 |
| Microcrystalline cellulose | 35 |
| Hydroxypropylmethylcellulose K4M | 25 |
| Hydroxypropylmethylcellulose K15M | 10 |
| Poly vinyl pyrollidone k 30 | 5 |
| Distilled water | Qs. |
| Magnesium stearate | 1.6 |
| Second Layer | |
| Bambuterol Hydrochloride | 10 |
| Starch | 25 |
| Lactose | 55 |
| Microcrystalline cellulose | 20 |
| Poly vinyl pyrollidone k 30 | 2.0 |
| Distilled water | Qs. |
| Magnesium stearate | 0.45 |

Procedure: Same as described in Example-I

1. A pharmaceutical tablet formulation comprising (i) a sustained release layer comprising at least one xanthine, and (ii) an immediate release layer comprising at least one β-2 adrenoreceptor against selected from the group consisting of long acting and short cting β-2 adrenoreceptor agoinists.

2. A pharmaceutical formulation according to claim 1, wherein said long acting β-2 adrenoreceptor agonist is selected from the group consisting of salmeterol, bambuterol and formoterol, or a pharmaceutically acceptable salt, solvate or physiologically functional derivative thereof.

3. A pharmaceutical formulation according to claim 2, wherein said short acting β-2 adrenoreceptor agonist is bambuterol hydrochloride.

4. A pharmaceutical formulation according to claim 1, wherein said short acting β-2 adrenoreceptor agonist is selected from the group consisting of pirbuterol acetate, bitolterol mesylate, procaterol and metaprolol sulfate.

5. A pharmaceutical formulation according to claim 1, wherein said xanthine is selected from the group consisting of theophylline, theobromine, aminophylline, doxophylline and enprofylline, or a pharmaceutically acceptable salt, solvate or physiologically functional derivative thereof.

6. A pharmaceutical formulation according to claim 5, wherein said xanthine is theophylline.

7. A pharmaceutical tablet formulation comprising (i) a sustained release layer comprising theophylline, and (ii) an immediate release layer comprising bambuterol hydrochloride.

8. A pharmaceutical formulation according to claim 1, wherein said sustained release layer further comprises a water soluble or water swellable polymeric release material.

9. A pharmaceutical formulation according to claim 8, wherein said water soluble or water swellable polymeric release material delays the release of the at least one xanthines from the sustained release layer for a predetermined period of time.

10. A pharmaceutical tablet formulation comprising (i) a sustained release layer comprising at least one xanthine, and (ii) an immediate release layer comprising at least one β-2 adrenoreceptor agonist selected from the group consisting of long acting and short acting β-2 adrenoreceptor agonists, further characterized in that a water soluble or water swellable polymeric release material is provided which delays the release of the at least one xanthines from the sustained release layer for a predetermined period of time.

11. A pharmaceutical formulation according to claim 1, which comprises:
(a) 40 to 55 weight % theophylline;
(b) 10 to 25 weight % water soluble or water swellable hydrophilic or lipophillic gel forming polymers;
(c) 10 to 30 weight % diluents; and
(d) 1 to 5 weight % binders.

12. A pharmaceutical formulation according to claim 8, wherein said water soluble or water swellable polymer is selected from the group consisting of polyvinylpyrrolidone, hydroxypropyl methyl cellulose, methyl cellulose, sodium carboxy methyl cellulose, cross linked carboxy methyl cellulose, hydroxy ethyl cellulose and hydroxy propyl cellulose.

13. A pharmaceutical formulation according to claim 12, wherein the polymeric material comprises hydroxypropyl methyl cellulose and polyvinylpyrrolidone.

14. A process of preparing a pharmaceutical formulation according to claim 1, which comprises providing at least one xanthine and at least one β-2 adrenoreceptor agonist selected from the group consisting of long acting and short acting β-2 adrenoreceptor agonists, formulating said at least one xanthine so as to provide said sustained release layer of said formulation and formulating said β-2 adrenoreceptor agonist so as to provide said immediate release layer of said formulation.

15. A method of treating a respiratory disease in a subject in need of treatment, which method comprises administering to the subject a pharmaceutical formulation according to claim 1.

16. A method according to claim 15, wherein the respiratory disease is asthma.

17. A method according to claim 16, wherein the respiratory disease is nocturnal asthma.

18. A method of making a medicament comprising providing at least one long acting β-2 adrenoreceptor agonist selected from the group consisting of long acting and short acting β-2 adrenoreceptor agonists, and at least one xanthine, and making a medicament for the treatment of respiratory disease, wherein said medicament comprises (i) a sustained release layer comprising said at least one xanthine, and (ii) an immediate release layer comprising said β-2 adrenoreceptor agonist.

19. A method of combining at least one long acting β-2 adrenoreceptor agonist selected from the group consisting of long acting and short acting β-2 adrenoreceptor agonists, and at least one xanthine, in the manufacture of a medicament for the treatment of asthma.

20. The method according to claim 19, wherein the medicament is in a form suitable for the treatment of nocturnal asthma.
